SEQUENCE LISTING (4 sequences)

SEQ ID NO: 1 - protein, 9 aa, synthetic construct
  REGION 1..9: note = R-Pro9-3 peptide
RRWIALWLR

SEQ ID NO: 2 - protein, 9 aa, synthetic construct
  REGION 1..9: note = R-Pro9-3D peptide
RRWIALWLR

SEQ ID NO: 3 - protein, 9 aa, synthetic construct
  REGION 1..9: note = Pro9-3 peptide
RLWLAIWRR

SEQ ID NO: 4 - protein, 9 aa, synthetic construct
  REGION 1..9: note = Pro9-3D peptide
RLWLAIWRR

(19) United States
(12) Patent Application Publication
(10) Pub. No.: US 2025/0019398 A1
(43) Pub. Date: Jan. 16, 2025

(54) NONAMERIC PEPTIDE HAVING EXCELLENT ANTIBACTERIAL ACTIVITY AGAINST GRAM-NEGATIVE BACTERIA AND ENANTIOMER THEREOF

(71) Applicant: KONKUK UNIVERSITY INDUSTRIAL COOPERATION CORP, Seoul (KR)

(72) Inventors: Yangmee KIM, Seoul (KR); Joon Hyeok CHOI, Seoul (KR); Hyeju LEE, Seoul (KR)

(73) Assignee: KONKUK UNIVERSITY INDUSTRIAL COOPERATION CORP, Seoul (KR)

(21) Appl. No.: 18/711,556

(22) PCT Filed: Nov. 4, 2022
(86) PCT No.: PCT/KR2022/017267

§ 371 Date: May 17, 2024

(30) Foreign Application Priority Data
Nov. 19, 2021 (KR) 10-2021-0160777

(51) Int. Cl. C07K 7/06; A61K 38/00; A61P 31/04

(52) U.S. Cl. CPC C07K 7/06; A61P 31/04; A61K 38/00

(57) ABSTRACT

A nonameric peptide represented by an amino acid sequence of SEQ ID NO: 1 or its enantiomeric peptide thereof according to the present invention is low in cytotoxicity and excellent in terms of proteolytic stability, antimicrobial activity, and anti-inflammatory activity and has a remarkable therapeutic effect on Gram-negative bacterial infectious diseases, sepsis, or septic shock. Accordingly, the peptides can be advantageously applied to an antimicrobial composition, an anti-inflammatory composition, and a pharmaceutical composition for prevention or treatment of Gram-negative bacterial infectious diseases, sepsis, or septic shock.

### CROSS REFERENCE TO RELATED APPLICATION(S)

This application is a 35 U.S.C. § 371 National Stage of International Patent Application No. PCT/KR2022/017267, filed Nov. 4, 2022, claiming benefit from Korean Patent Application No. 10-2021-0160777, filed Nov. 19, 2021, the disclosures of which are incorporated herein in their entirety by reference, and priority is claimed to each of the foregoing.

### TECHNICAL FIELD

The present invention relates to a nonameric peptide and its enantiomeric peptide thereof, which have excellent antimicrobial activity against Gram-negative bacteria, and more particularly, to a nonameric antimicrobial peptide represented by an amino acid sequence of SEQ ID NO: 1 or its enantiomeric antimicrobial peptide thereof, and an antimicrobial composition, an anti-inflammatory composition, and a pharmaceutical composition for the prevention or treatment of Gram-negative bacterial infectious diseases, sepsis, or septic shock, which include the antimicrobial peptide as an active ingredient.

Meanwhile, the present application was supported by the following national research and development project.

### NATIONAL RESEARCH AND DEVELOPMENT PROJECT SUPPORTING PRESENT INVENTION

[Project identification No.] 1711107062
[Project No.] 2020R1A2C2005338
[Government department] Ministry of Science and ICT
[Research Management Organization] National Research Foundation of Korea
[Project Name] Basic research project (mid-career researchers—core research)
[Project Title] Understanding the mechanism of virulence and discovering inhibitory antibiotics through structure-function studies of specific fatty acid synthesis proteins of carbapenem-resistant Acinetobacter bacteria
[Supervising Institution] Konkuk University
[Research Period] Mar. 1, 2021 to Feb. 28, 2022

### BACKGROUND

In Gram-negative infections, lipopolysaccharide (LPS), which is produced from the outer membrane of bacteria and act as an endotoxin, activates immune cells to cause severe inflammation, organ failure, and even death through an unregulated immune response.

Six pathogens (ESKAPE) identified by the World Health Organization (WHO) as highly resistant to antibiotics are Enterococcus faecium, Staphylococcus aureus, Klebsiella pneumoniae, Acinetobacter baumannii, Pseudomonas aeruginosa, and Enterobacter species. Among them, Gram-negative A. baumannii is an important opportunistic infectious pathogen with high mortality in hospitals and infects about 1 million people each year, and 44% of clinical isolates are multidrug-resistant (MDR) A. baumannii. The most common resistance mechanisms in these isolates include penicillin-binding protein mutation, porin loss, aminoglyco-side-modifying enzyme mutation, efflux pump overexpression, and the like.

Primary therapeutic agents against A. baumannii include β-lactam or carbapenem antibiotics such as doripenem, imipenem, and meropenem. However, the emergence of carbapenem-resistant A. baumannii (CRAB) bacteria, which are resistant to carbapenem antibiotics due to β-lactamase hydrolysis and carbapenemase overproduction, has made it more difficult to treat infections. The resistance mechanism to colistin and/or tigecycline in CRAB bacteria is associated with structural changes in LPS through mutations of genes encoding lipoxygenase and polymyxin resistance-associated response regulator (pmr)-A/B. Further, antibiotic resistance may induce the formation of biofilms, which allow secreted substances such as extracellular matrix polysaccharides, proteins and DNA to be deposited on biological or abiotic surfaces, thereby increasing the toxicity and antibiotic resistance of CRAB isolates in immunocompromised patients. Currently, the development of drugs to treat CRAB infections is very limited.

Pathogenic stimuli may lead to the progression of host immune responses to systemic inflammatory response syndrome (SIRS), increasing the severity of infection and ultimately inducing the pathological process of sepsis.

Sepsis is a multifaceted syndrome caused by an abnormal immune response to bacterial infections, thereby culminating in excessive inflammation, organ dysfunction, and even death (Van Der Poll T. et al., Nat. Rev. Immunol., 17:407-420, 2017; Rudd K. E. et al., Crit. Care, 22:1-11, 2018).

According to a nationwide cohort study, the incidence of sepsis cases in Korea has increased, with approximately 233.6 cases per 100,000 people and a 22.6% mortality rate in 2016. In particular, infections by Gram-negative bacteria require an urgent solution because the outer cell membrane of bacteria consists of lipopolysaccharide (LPS), which not only triggers abnormal stages of the inflammatory response but also hinders the development of new antibiotics against resistant infections (Delcour A. H., Biophys. Acta (BBA) Proteins Proteom, 1794:808-816, 2009; Tucureanu M. M. et al., Int. J. Nanomed, 13:63-76, 2017).

Several studies in the relevant field have revealed that the excessive activation of toll-like receptors (TLRs) by invading pathogen or pathogenic molecules, such as LPS, Pam2CSK4, or Pam3CSK4, plays an important role in sepsis pathogenesis (Cohen J, Nature, 420:885-891, 2002). Despite dramatic advances in sepsis pathophysiology and treatment procedures, no therapeutic agents for Gram-negative sepsis have been approved to date. Therefore, the search for a novel class of antibiotics that can combat and overcome symptoms associated with sepsis is urgently required.

Antimicrobial peptides (AMPs) are part of the first line of defense in all living host species and have emerged as a new class of antibiotics for treating infections. AMPs form a component of the innate immune defense, exhibit broad-spectrum potential as small molecule antibiotics, and have emerged as an alternative material to naturally occurring antibiotics through more than 40 years of research on peptides. In addition, since AMPs have various structures and modes of action with a wide range of antimicrobial activities, the development of new AMPs is being investigated as a promising strategy to combat multidrug-resistant bacteria (Wimley W. C. et al., J. Membr. Boil., 239:27-34, 2011; Narayana J. L. et al., Peptides, 72:88-94, 2015; Hollmann A. et al., Front. Chem., 6:204, 2018).

Although about 3,000 or more AMPs have been identified to date, only few of them are received FDA-approval. Among the FDA-approved AMPs are colistin, gramicidin D, daptomycin, vancomycin, oritavancin, dalbavancin, and telavancin, which are currently available on the market or in clinical development (Steckbeck J. D. et al., Boil. Ther., 14:11-14, 2013; Wang G., Li X. et al., Nucleic Acids Res., 44:D1087-D1093, 2015). Although they have different structures and sequence motifs, they share a broad range of antimicrobial actions, and although the detailed mechanisms of the antimicrobial activity of AMPs have not been fully understood, they have been identified to be related to bacterial cell membrane penetration or intracellular killing.

Many AMPs have potential resistance, immunomodulatory abilities, and a broad spectrum of bactericidal activities such as anti-inflammatory, anti-biofilm and anti-viral effects, which are not commonly found in conventional antibiotics. To address the susceptibility of AMPs to proteolysis, various peptidomimetics have been created, incorporating strategies such as D-amino acids, unnatural amino acids, peptide backbone modifications, cyclization, triazole modifications, and secondary structure-inducing templates.

However, controlling selectivity, improving stability, and reducing cytotoxicity-mediated side effects are required for the development of potent AMPs, and short peptide analogs designed based on natural AMPs with high antimicrobial activity can reduce production costs and induce low immunogenicity, so the short peptide analogs can be developed as novel candidates for therapeutic application fields. Although protaetiamycine is an insect defensin consisting of 43 amino acids and has strong antimicrobial activity against both Gram-negative and Gram-positive bacteria, it is a 43-mer AMP having a long residue sequence, making it difficult to be commercialized due to its low economic feasibility. The inventors of the present invention had designed a nonameric peptide (Ala22 to Gly30) from protaetiamycine, which is an insect defensin, in a previous study. The nonameric peptides Pro9-3 and Pro9-3D (enantiomeric peptides) developed therefrom have excellent antimicrobial activity but exhibited high cytotoxicity (Shin S. et al., J. Pept. Sci., 15:559-568, 2009; Lee, E. et al., J. Pept. Sci., 17, 675-682, 2011; Korean Patent No. 10-1193398; Korean Patent No. 10-1247350; Korean Patent No. 10-1420849; Korean Patent No. 10-1599587).

### SUMMARY OF THE INVENTION

Thus, in the present invention, as a result of intensive studies to develop a potent and short peptide with high selectivity and low cytotoxicity to treat Gram-negative bacterial infections, the peptide sequences of Pro9-3 and Pro9-3D, two peptides designed in the previous study, were reversed, the cationic nature of the N-terminus was increased by allowing the two peptides to have two sequential Arg residues at the N-terminus, and D-amino acids were introduced, thereby designing new peptides (R-Pro9-3 and R-Pro9-3D). It was confirmed that the designed peptides exhibited rapid and effective antimicrobial activity against Gram-negative bacteria, particularly carbapenem-resistant Gram-negative bacteria by increasing bacterial cell membrane permeability, and were excellent in terms of proteolytic stability and cytotoxicity. Furthermore, the inventors of the present invention evaluated the anti-inflammatory activity of these peptides, and confirmed that R-Pro9-3D has an excellent therapeutic effect on sepsis caused by Gram-negative bacterial infections and can be applied as a peptide antibiotic, thereby completing the present invention.

The present invention is directed to providing a nonameric peptide represented by an amino acid sequence of SEQ ID NO: 1 or its enantiomeric antimicrobial peptide thereof, which has antimicrobial activity, anti-inflammatory activity, and prevents Gram-negative bacterial infections, and a use of the antimicrobial peptide for the prevention or treatment of sepsis or septic shock.

### Technical Solution

One aspect of the present invention provides a nonameric peptide represented by an amino acid sequence of SEQ ID NO: 1 or its enantiomeric peptide thereof.

In the present invention, the nonameric peptide or its enantiomeric peptide thereof may be characterized by being an antimicrobial peptide.

In the present invention, the enantiomeric peptide may be characterized in that all of the amino acids of the amino acid sequence of SEQ ID NO: 1 are substituted with D-type amino acids.

In the present invention, the bacteria may be characterized by being Gram-negative bacteria.

In the present invention, the Gram-negative bacteria may be characterized by being multidrug-resistant (MDR) Gram-negative bacteria.

In the present invention, the Gram-negative bacteria may include one or more selected from the group consisting of Escherichia coli, Acinetobacter baumannii, Pseudomonas aeruginosa, and Klebsiella pneumoniae.

In the present invention, the multidrug-resistant (MDR) Gram-negative bacteria may be characterized by being carbapenem-resistant.

In the present invention, the nonameric peptide or its enantiomeric peptide thereof may be characterized by having low hemolytic activity and low toxicity on mammalian cells.

In the present invention, the nonameric peptide or its enantiomeric peptide thereof may be characterized by increasing bacterial cell membrane permeability to damage bacterial cell membranes.

In the present invention, the nonameric peptide or its enantiomeric peptide inhibits the activity of lipopolysaccharide (LPS), which is a component of the outer membrane of Gram-negative bacteria and acts as an endotoxin, via binding to LPS.

In the present invention, the nonameric peptide or its enantiomeric peptide thereof may be characterized by suppressing the formation of a bacterial biofilm.

In the present invention, the enantiomeric peptide may be characterized by having proteolytic stability with resistance to proteases.

In the present invention, the nonameric peptide or its enantiomeric peptide thereof may be characterized by suppressing inflammation induced by LPS.

In the present invention, the nonameric peptide or its enantiomeric peptide thereof may be characterized by being used for the treatment of Gram-negative sepsis or septic shock.

Another aspect of the present invention provides an antimicrobial composition including the peptide as an active ingredient.

Still another aspect of the present invention provides an anti-inflammatory composition including the peptide as an active ingredient.

Yet another aspect of the present invention provides a pharmaceutical composition for preventing or treating Gram-negative bacterial infectious diseases, including the peptide as an active ingredient.

In the present invention, the Gram-negative bacterial infectious disease may be characterized by being Gram-negative sepsis or septic shock.

Yet another aspect of the present invention provides a quasi-drug composition for preventing or ameliorating Gram-negative bacterial infectious diseases, including the peptide as an active ingredient.

In the present invention, the Gram-negative bacterial infectious disease may be characterized by being Gram-negative sepsis or septic shock.

Yet another aspect of the present invention provides a food composition for preventing or ameliorating Gram-negative bacterial infectious diseases, including the peptide as an active ingredient.

In the present invention, the Gram-negative bacterial infectious disease may be characterized by being Gram-negative sepsis or septic shock.

Yet another aspect of the present invention provides a method for preventing or treating Gram-negative bacterial infectious diseases, the method including: administering a pharmaceutical composition including the peptide as an active ingredient to a patient with a Gram-negative bacterial infectious disease.

In the present invention, the Gram-negative bacterial infectious disease may be characterized by being Gram-negative sepsis or septic shock.

Yet another aspect of the present invention provides a use of the peptide for preventing or treating Gram-negative bacterial infectious diseases.

In the present invention, the Gram-negative bacterial infectious disease may be characterized by being Gram-negative sepsis or septic shock.

Yet another aspect of the present invention provides a use of the peptide for preparing a drug for preventing or treating Gram-negative bacterial infectious diseases.

In the present invention, the Gram-negative bacterial infectious disease may be characterized by being Gram-negative sepsis or septic shock.

### Advantageous Effects

The nonameric peptide represented by an amino acid sequence of SEQ ID NO: 1 or its enantiomeric peptide thereof has lower cytotoxicity and excellent proteolytic stability compared to a nonameric peptide represented by an amino acid sequence of SEQ ID NO: 3 or its enantiomeric peptide thereof, exhibits excellent antimicrobial activity by effectively penetrating bacterial cell membranes, and effectively kills biofilms.

Further, the nonameric peptide represented by the amino acid sequence of SEQ ID NO: 1 or its enantiomer thereof inhibits LPS, which is a main component of the outer membrane of Gram-negative bacteria and acts as an endotoxin, via strong binding to lipopolysaccharide (LPS).

Therefore, the nonameric peptide represented by the amino acid sequence of SEQ ID NO: 1 or its enantiomeric peptide thereof has an advantage by demonstrating remarkable anti-inflammatory effects and can be used as a new peptide antibiotic for treating infectious diseases caused by antibiotic-resistant Gram-negative bacteria.

Furthermore, the nonameric peptide represented by the amino acid sequence of SEQ ID NO: 1 or its enantiomeric peptide thereof ameliorates inflammatory insults in a septic mouse model, and exhibits a protective effect against organ dysfunction in septic mice, and thus can be usefully applied as a new peptide antibiotic for the effective treatment of sepsis or septic shock.

### BRIEF DESCRIPTION OF THE DRAWINGS

FIG. 1 relates to the helical wheel projection views of Pro9-3 (FIG. 1A) and R-Pro9-3 (FIG. 1B) analyzed using the HeliQuest program (http://heliquest.ipmc.cnrs.fr/cgi-bin/ComputParams.py). Positively charged amino acid residues are shown in blue, negatively charged residues are shown in red, hydrophobic residues are shown in yellow, and alanine is shown in gray, confirming that the peptide exhibits amphipathic properties. Arrows, N and C represent the helical hydrophobic moments, an N-terminal region and a C-terminal region, respectively.

FIG. 2 relates to the antibacterial mechanism of peptides. FIG. 2A shows a displacement assay using BODIPY-TR-cadaverine fluorescent dye showing the LPS binding affinity of a peptide, FIG. 2B shows an LAL assay showing the LPS neutralization ability of the peptide and LL-37 control (FIG. 2B), and FIG. 2C shows the concentration-dependent depolarization ability by peptides using intact CRAB bacteria. Statistical analysis was performed using two-way ANOVA with a Dunnett's comparison test. Each value is shown as the mean±SEM of three independent experiments and is statistically significant at *P<0.05, **P<0.01, and ***P<0.001. ns indicates not significant.

FIG. 3 relates to the biofilm inhibiting properties of peptides. The anti-biofilm activity of peptides (0-32 μM, 16 h) against A. baumannii (FIG. 3A) or CRAB 12006 (FIG. 3B) was determined using crystal violet staining. Melittin, meropenem and imipenem were used as controls. Statistical analysis was performed using two-way ANOVA with a Dunnett's comparison test. Each value is shown as the mean±SEM of three independent experiments and is statistically significant at *P<0.05, **P<0.01; and ***P<0.001. ns indicates not significant.

FIG. 4 shows the circular dichroism spectra for peptides at a concentration of 100 μM. CD(Circular Dichroism) spectra were scanned 10 times in (A) an aqueous solution and (B) 50 mM dodecylphosphocholine (DPC) micelles. In FIG. 4, the double negative maxima at 205 and 220 nm are the characteristics of an alpha helical structure.

FIG. 5 relates to the in vitro cytotoxicity of the peptides. A dose-response curve shows the hemolytic activity of the peptide on sheep red blood cells (sRBCs) (FIG. 5A). FIG. 5 shows the concentration-dependent (0-100 μM) toxic effects induced by peptides on (B) RAW 264.7 murine macrophage cells and (C) human kidney (HK)-2 cells for 24 hours. Melittin was used as a control. Statistical analysis was performed using two-way ANOVA with a Dunnett's comparison test. Each value is shown as the mean±SEM of three independent experiments and is statistically significant at *P<0.05, **P<0.01; and ***P<0.001. ns indicates not significant.

FIG. 6 relates to the effect of the peptide on suppressing the inflammatory response in RAW264.7 cells stimulated with LPS. FIG. 6 shows the concentration-dependent inhibitory effects of peptides (0 to 50 μM) on (A) nitrite production and inflammatory cytokine (B) TNF-α and (C) IL-6 production in RAW264.7 cells stimulated with LPS (20 ng/mL) for 16 hours. Data was obtained in triplicate and is shown as mean±SEM.

FIG. 7 relates to the non-toxic effect in vivo, which is essential for R-Pro9-3D to be used as a therapeutic agent for Gram-negative bacterial infections or sepsis. (A) AST, (B) ALT and (C) BUN levels of mice treated with R-Pro9-3D (1 mg/kg and 5 mg/kg) for 24 hours were confirmed, and control mice were injected intraperitoneally with only a PBS solution. Data was obtained in triplicate and is shown as mean±SEM.

FIGS. 8A and 8B relate to the effect of R-Pro9-3D on alleviating septic shock in mice infected with E. coli K1. (A) inhibition of bacterial growth by R-Pro9-3D in lung, liver and kidney tissues of septic mice, (B) effect of R-Pro9-3D on reducing circulating endotoxin (LPS) levels in the serum of septic mice, effect of R-Pro9-3D on reducing (C) AST, (D) ALT, and (E) BUN concentrations in the serum of septic mice, effect of R-Pro9-3D on reducing (F) TNF-α and (G) IL-6 levels in serum tissues of septic mice, and effect of R-Pro9-3D on reducing (H) TNF-α and (I) IL-6 levels in lung tissue were confirmed. Graphs are shown as mean±SEM (5 mice/group). (J) confirmed the therapeutic effect of sepsis by suppressing lung damage in the lung tissue of septic mice infected with E. coli K1.

### DETAILED DESCRIPTION

Unless otherwise defined, all technical and scientific terms used herein have the same meaning as commonly understood by a person with ordinary skill in the art to which the present invention pertains. In general, the nomenclature used herein is well known and commonly used in the art.

Throughout the present specification, typical one- and three-letter codes for naturally occurring amino acids are used, as well as commonly accepted three-letter codes are used for other amino acids such as α-aminoisobutyric acid (Aib) and N-methylglycine (Sar) are used. Further, amino acids referred to as abbreviations in the present invention were described according to IUPAC-IUB nomenclature as follows:
alanine: A, arginine: R, asparagine: N, aspartic acid: D, cysteine: C, glutamic acid: E, glutamine: Q, glycine: G, histidine: H, isoleucine: I, leucine: L, lysine: K, methionine: M, phenylalanine: F, proline: P, serine: S, threonine: T, tryptophan: W, tyrosine: Y and valine: V.

The emergence of antibiotic-resistant (for example, carbapenem-resistant) Gram-negative bacteria is increasing, and Gram-negative bacteria (for example, carbapenem-resistant A. baumannii (CRAB)) induce untreatable antibiotic-resistant infections, and thus is becoming a problem worldwide. Currently, new drugs and techniques currently in development and clinical trials to treat CRAB infections are very limited.

Therefore, the present invention proposes antimicrobial peptides (AMPs) with broad-spectrum bactericidal and immunomodulatory properties as a potential alternative to existing antibiotics as a new therapeutic approach to prevent the spread of antibiotic-resistant A. baumannii infections. Although the clinical application of AMPs is limited by their propensity for enzymatic proteolysis, peptides with D-amino acid substitutions are resistant to proteolysis in vivo, thus securing maximum bioavailability and therapeutic efficacy.

In a previous study, two nonameric peptides, Pro9-3 and Pro9-3D (enantiomeric peptides) were designed based on the structure-activity relationship of insect defensin, but showed high cytotoxicity in mammalian cells.

Thus, in the present invention, R-Pro9-3 and R-Pro9-3D were designed by reversing the sequences of the two nonameric peptides designed in the previous study and introducing D-amino acids to increase the stability of the peptides, and it was confirmed that the designed peptides penetrated bacterial membranes to exert better antimicrobial activity against Gram-negative bacteria, particularly carbapenem-resistant Gram-negative bacteria, and exhibited excellent proteolytic stability and low cytotoxicity. Furthermore, the present invention was completed by confirming that the peptides have excellent anti-inflammatory activity and exhibit a sepsis-suppressing effect.

The present invention provides a nonameric peptide represented by an amino acid sequence of SEQ ID NO: 1 or its enantiomeric peptide thereof.

In the present invention, the nonameric peptide or its enantiomeric peptide thereof may be characterized by being an antimicrobial peptide.

In the present invention, the nonameric peptide or its enantiomeric peptide thereof has the reverse sequence of a nonameric peptide represented by an amino acid sequence of SEQ ID NO: 3 or its enantiomeric peptide thereof.

As used herein, the term “antimicrobial” refers to the ability to reduce, prevent, suppress or eliminate the growth or survival of microorganisms at a certain concentration, and may preferably refer to “antibacterial or anti-bacterial.”

In the present invention, the enantiomeric peptide is characterized in that all of the amino acids of the amino acid sequence of SEQ ID NO: 1 are substituted with D-type amino acids.

The inventors of the present invention had designed four nonameric peptides (Ala22 to Gly30) from protaetiamycine, which is a 43-mer insect defensin, in a previous study. The third analog of the four peptides, Pro9-3 (RLWLAIWRR-NH2, SEQ ID NO: 3), and its enantiomeric peptide Pro9-3D showed potent antimicrobial and anti-inflammatory activities, but also exhibited high cytotoxicity to mammalian cells (Shin S. et al., J. Pept. Sci., 15:559-568, 2009; Lee E. et al., J.Pept. Sci., 17:675-682, 2011).

In the present invention, in order to enhance antimicrobial activity against Gram-negative bacteria and improve bacterial cell selectivity, a reverse 9-meric peptide was prepared using the amino acid sequence of Pro9-3 in reverse and named “R-Pro9-3.” In addition, in order to increase the stability of the peptide, “R-Pro9-3D” in which the amino acids of R-Pro9-3 were substituted with D-type amino acids was additionally synthesized (Table 1).

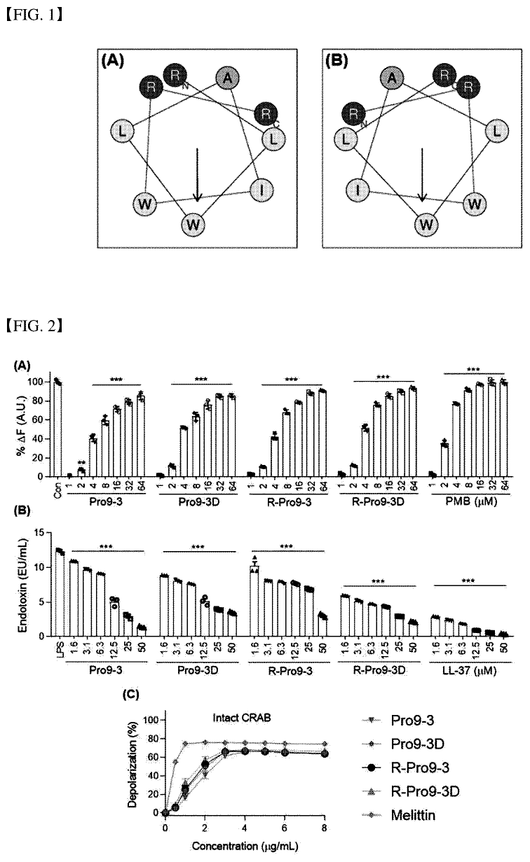

In a specific exemplary embodiment of the present invention, helical-wheel diagram analysis was performed on the parent peptides Pro9-3 and R-Pro9-3. As shown in FIG. 1, it was confirmed that the helical-wheel diagrams of the parent peptides Pro9-3 and R-Pro9-3 were designed to effectively penetrate the cell membrane of Gram-negative bacteria, which is negatively charged and has amphipathic properties, by confirming that the lower hydrophobic surface is much larger than the upper hydrophilic surface, but exhibits amphipathic properties, is equally highly cationic, and has the same hydrophobicity (0.776).

In the present invention, the bacteria may be characterized by being Gram-negative bacteria, but is not limited thereto.

In the present invention, the Gram-negative bacteria may be characterized by being multidrug-resistant (MDR) Gram-negative bacteria, but is not limited thereto.

In the present invention, the Gram-negative bacteria may include one or more selected from the group consisting of Escherichia coli, Acinetobacter baumannii, Pseudomonas aeruginosa, and Klebsiella pneumoniae, but is not limited thereto.

In the present invention, the multidrug-resistant (MDR) Gram-negative bacteria may be characterized by being carbapenem-resistant, but is not limited thereto.

In a specific exemplary embodiment of the present invention, the antimicrobial activity of R-Pro9-3 and R-Pro9-3D was determined. As a result of determining the antimicrobial activity of Pro9-3, Pro9-3D, R-Pro9-3, and R-Pro9-3D against representative Gram-negative bacterial standard bacteria and MDR Gram-negative bacterial strains, as shown in Table 2, all the peptides showed antimicrobial activity similar to melittin, which is known as a potent peptide antibiotic. In particular, it was confirmed that R-Pro9-3D show an excellent bactericidal efficacy against all standard Gram-negative bacteria and all Gram-negative MDR bacteria when compared with the other nonameric peptides (Pro9-3, Pro9-3D, R-Pro9-3). In addition, it was confirmed that R-Pro9-3D showed a higher relative selective index against Gram-negative bacteria than the other peptides, and the relative selective index was higher in the order of R-Pro-9-3D, Pro9-3D, R-Pro9-3, and Pro9-3 (34.6, 26.5, 8.0, and 6.3, respectively). Therefore, it was confirmed that R-Pro9-3D showed excellent bacterial selectivity compared to the other peptides.

As used herein, the term multidrug-resistant (MDR) Gram-negative bacteria (or resistant bacteria) refers to bacteria that exhibit resistance to antibiotics as a result of continued use of drugs in order to treat or prevent any disease and complications thereof or any bacterial disorder and complications thereof. Examples of the antibiotics include vancomycin, cephalosporin, quinolone and fluoroquinolone, penicillin, beta-lactamase inhibitors, carbapenem, monobactam, macrolide and lincosamine, glycopeptide, rifampin, oxazolidinone, tetracycline, aminoglycoside, streptogramin and sulfonamide, and the like, and antibiotic-resistant bacteria also exhibit resistance even when treated with the antibiotics listed above, so that the disease persists in an individual.

In the present invention, the nonameric peptide or its enantiomeric peptide thereof may be characterized by having low hemolytic activity and low toxicity on mammalian cells infected with Gram-negative bacteria, but is not limited thereto.

In the present invention, the cells may be eukaryotic cells, and mammalian cells may be preferably animal or human cells.

In a specific exemplary embodiment of the present invention, as a result of confirming whether the peptides induce cytotoxic effects, as shown in FIG. 5A, R-Pro9-3, R-Pro9-3D, Pro9-3, and Pro9-3D peptides induced 1 to 5% hemolysis of sRBCs even at 100 μM, whereas melittin showed 100% hemolysis at a concentration of 25 μM.

Furthermore, as a result of confirming the cytotoxicity of the peptides to a murine macrophage cell line (RAW264.7), as shown in FIGS. 5B and 5C, both R-Pro9-3 and Pro9-3 were found to be non-toxic even at a concentration up to 100 μM compared to melittin, but have lower antimicrobial activity. In contrast, Pro9-3D exhibited a toxicity of 44.5% and 22.9%, respectively, in RAW264.7 and human kidney (HK)-2 cells at a concentration of 100 μM, and R-Pro9-3D exhibited 2-fold lower toxicity of 20.3% and 9.6%. That is, although Pro9-3D showed high antimicrobial activity against MDR Gram-negative bacteria, it was confirmed that it cannot be used as an effective peptide antibiotic because it is highly toxic to mammalian cells.

In the present invention, the nonameric peptide or its enantiomeric peptide thereof may exhibit antimicrobial activity by increasing bacterial cell membrane permeability to damage bacterial cell membranes.

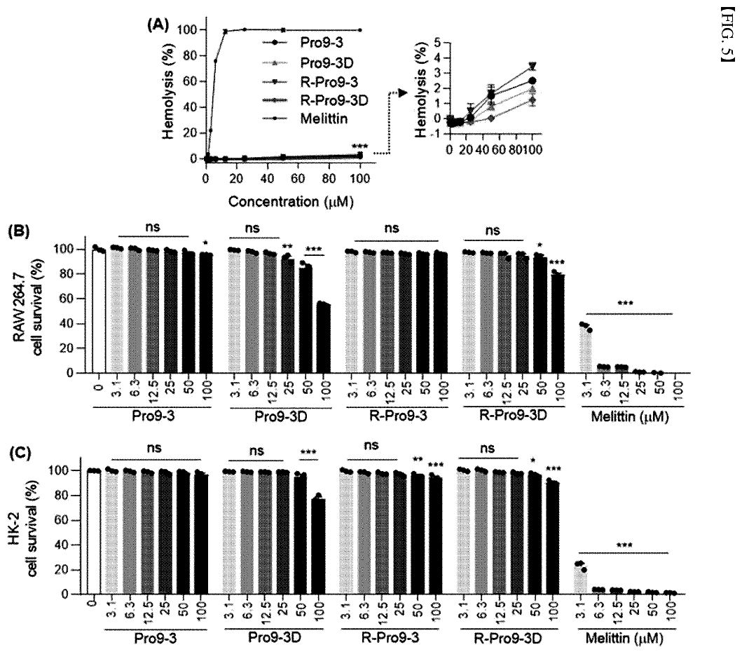

In a specific exemplary embodiment of the present invention, as a result of analyzing the secondary structure of the peptide in an environment such as an aqueous solution and a membrane by analyzing the CD spectra, as shown in FIG. 4, it was confirmed that all the peptides exhibited unaligned structures in an aqueous solution, but showed a change from dodecylphosphocholine (DPC) micelles to alpha-helical structures, and would effectively penetrate the bacterial cell membrane by having an amphipathic alpha-helical structure.

That is, the nonameric peptide or its enantiomeric peptide thereof may maintain high antimicrobial activity similar to melittin due to its high cationicity, and may effectively interact with bacterial cell membranes to cause fatal damage to bacteria, thereby improving antimicrobial activity.

In the present invention, the nonameric peptide or its enantiomeric peptide thereof may have the ability to neutralize endotoxins or ability to remove endotoxins in vitro and/or in vivo.

That is, the nonameric peptide or its enantiomeric peptide thereof may be characterized by binding to lipopolysaccharide (LPS) that acts as an endotoxin to suppress the activity of lipopolysaccharide (LPS).

In a specific exemplary embodiment of the present invention, the antibacterial mechanism of the peptide was investigated using membrane permeability experiments. LPS is an outer membrane component of Gram-negative bacteria which is attached to innate immune receptors (for example, toll-like receptors (TLRs)) which is critical in infectious disease. For example, an amphipathic peptide R-Pro9-3D can target LPS more effectively, which is a component of the outer membrane of Gram-negative bacteria and with amphipathic and anionic properties, through electrostatic interactions.

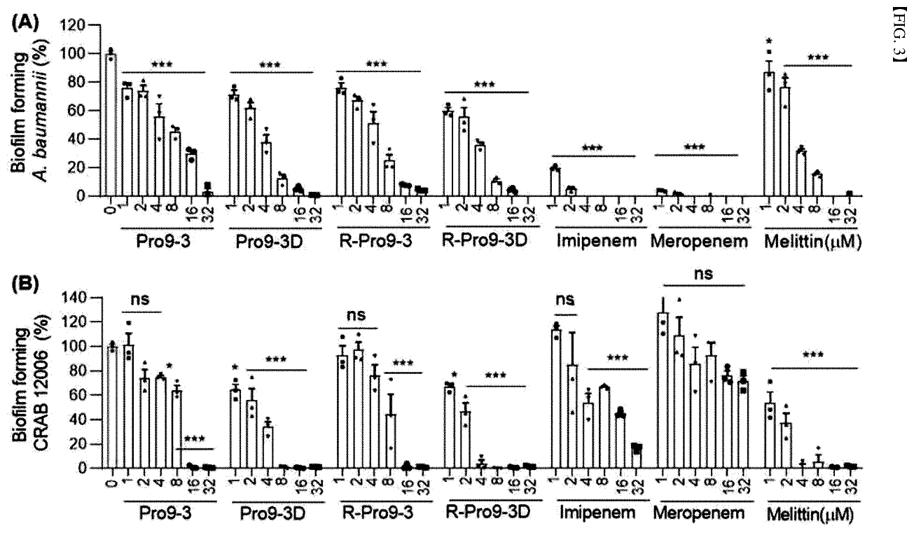

In a specific exemplary embodiment of the present invention, as a result of confirming the binding strength with LPS and the ability of the peptides to neutralize LPS, as shown in FIG. 2, it was confirmed that all the peptides interacted significantly with LPS in a concentration-dependent manner. As shown in FIG. 2A, it was confirmed that Pro9-3, Pro9-3D, R-Pro9-3 and R-Pro9-3D peptides bind to LPS with high binding strength similar to polymyxin B, which is a positive control and is known to have excellent binding strength to LPS. As shown in FIG. 2B, it was confirmed that the peptides exhibited excellent LPS neutralizing ability, although it was weaker than LL-37 which is a positive control and is known to have strong LPS neutralizing ability. As shown in FIG. 2C, Pro9-3, Pro9-3D, R-Pro9-3 and R-Pro9-3D peptides showed excellent depolarization ability against intact CRAB bacteria, confirming the antibacterial mechanism targeting the bacterial cell membrane.

In the present invention, the nonameric peptide or its enantiomeric peptide thereof may be characterized by suppressing the formation of a bacterial biofilm.

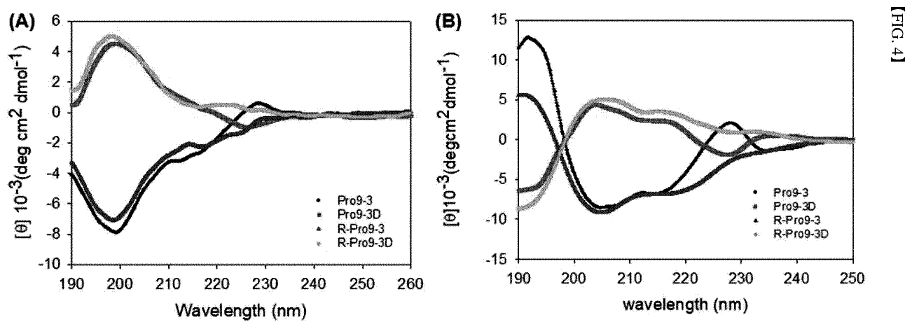

In a specific exemplary embodiment of the present invention, as a result of confirming the biofilm inhibitory effects of Pro9-3, R-Pro9-3, and R-Pro9-3D, as shown in FIG. 3, it was confirmed that all the peptides suppressed the biofilm formation in a concentration-dependent manner. In particular, R-Pro9-3D suppressed the biofilm formation by A. baumannii and CRAB strains most effectively compared to other peptides including melittin.

In the present invention, the enantiomeric peptide may be characterized by having proteolytic stability with resistance to proteases.

In a specific exemplary embodiment of the present invention, as a result of confirming the stability of R-Pro9-3 and R-Pro9-3D against endogenous proteases, as shown in Table 3, Pro9-3 and R-Pro9-3 were unable to suppress bacterial growth in the presence of the protease, whereas its enantiomeric peptides Pro9-3D and R-Pro9-3D maintained antimicrobial activity regardless of the presence of the protease, and suppressed the growth of E. coli and A. baumannii similarly to the absence of the protease. The above results imply that R-Pro9-3D exhibits substantial protease resistance to trypsin and chymotrypsin.

The main causes of biofilm formation by MDR bacteria are antibiotic resistance and bacterial susceptibility to proteolytic cleavage. In the present invention, it was found that R-Pro9-3D was completely undamaged under various digestion conditions and exhibited considerable antimicrobial activity against Gram-negative bacteria like Pro9-3D, and R-Pro9-3D exhibited more effective antimicrobial activity against A. baumannii than Pro9-3D and had improved proteolytic stability.

In the present invention, the nonameric peptide or its enantiomeric peptide thereof may suppress inflammation induced by LPS, but is not limited thereto.

In the present invention, the nonameric peptide or its enantiomeric peptide thereof may be characterized by being used for the treatment of Gram-negative sepsis or septic shock.

The septic shock is also called sepsis shock, and is a case where sepsis is accompanied by hypotension.

In another aspect, the present invention relates to an antimicrobial composition including the peptide as an active ingredient.

In still another aspect, the present invention relates to an anti-inflammatory composition including the peptide as an active ingredient.

In the present invention, the inflammation may be characterized by being inflammation induced by LPS.

LPS released by pathogens stimulates host immune cells such as macrophages and fibroblasts to produce cytokines capable of inducing sepsis-related diseases.

In a specific exemplary embodiment of the present invention, as a result of confirming the anti-inflammatory efficacy of the peptide in RAW264.7 cells stimulated with LPS, as shown in FIG. 6, it was confirmed that when treated with the peptide, (A) NO, (B) TNF-α, and (C) IL-6 levels were effectively reduced. Furthermore, it was confirmed that Pro9-3D and R-Pro9-3D, which are enantiomers of Pro9-3 and R-Pro9-3, more effectively suppressed the production of NO, TNF-α, and IL-6 than Pro9-3 and R-Pro9-3. These results indicate that R-Pro9-3D may efficiently downregulate LPS-induced cytokine levels.

In the present invention, it was confirmed that R-Pro9-3D has excellent antimicrobial activity and has excellent resistance to proteolytic enzymes, endotoxin neutralization/removal, biofilm formation inhibiting efficacy, and anti-inflammatory activity.

In yet another aspect, the present invention relates to a pharmaceutical composition for preventing or treating Gram-negative bacterial infectious diseases, including the peptide as an active ingredient.

In the present invention, the Gram-negative bacterial infectious disease may be characterized by being Gram-negative sepsis or septic shock.

In the present invention, in particular, R-Pro9-3D has excellent preventive or therapeutic effects on sepsis/septic shock, and may improve the function of organs damaged by endotoxins.

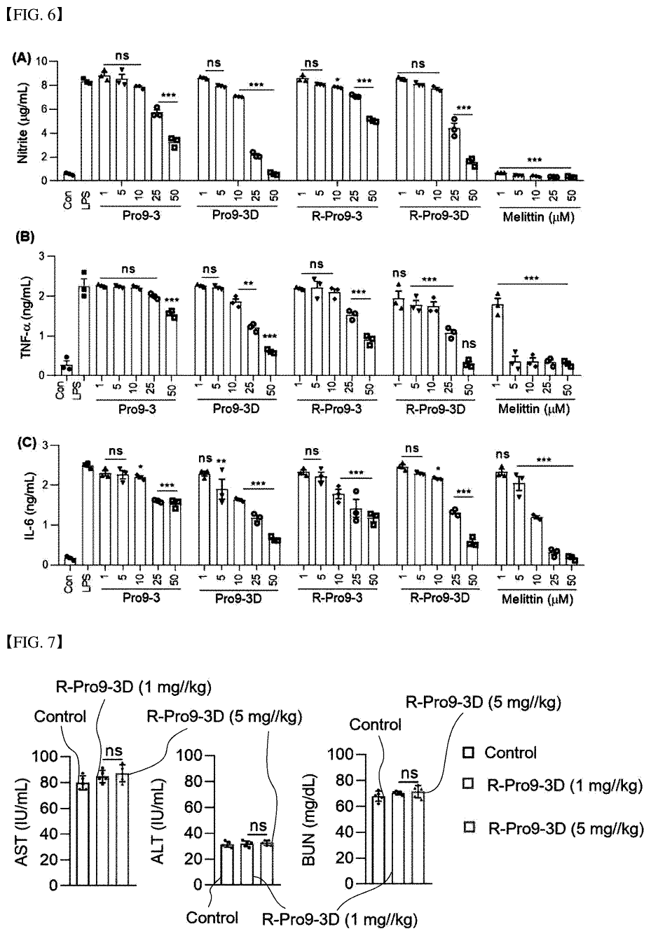

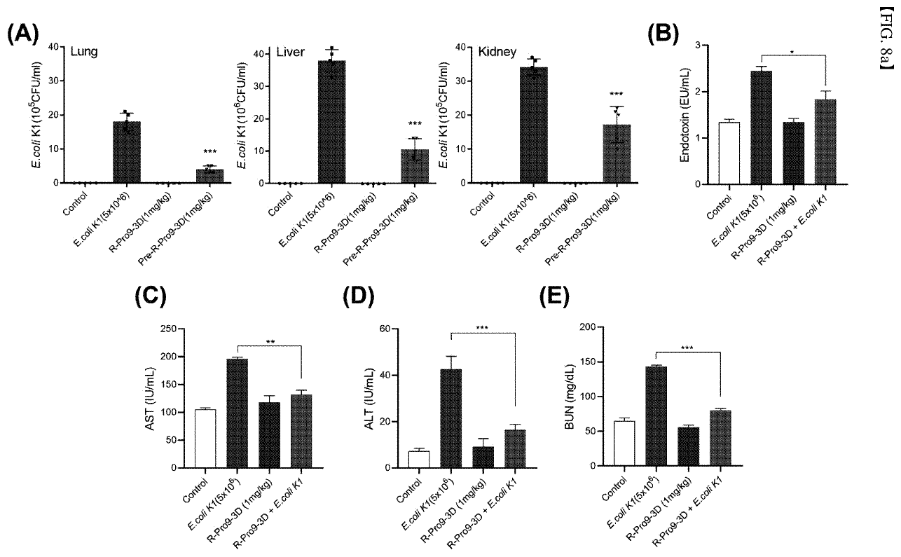

In a specific exemplary embodiment of the present invention, as a result of investigating the safety of R-Pro9-3D in mice in order to confirm the efficacy of R-Pro9-3D as a therapeutic agent for Gram-negative bacterial infections or as a therapeutic agent for sepsis, as shown in FIG. 7, it was confirmed in the present invention that R-Pro9-3D is safe even at high doses because no clinically significant levels of AST, ALT and BUN in treated mice were observed when low and high doses (1 mg/kg and 5 mg/kg) of R-Pro9-3D were administered.

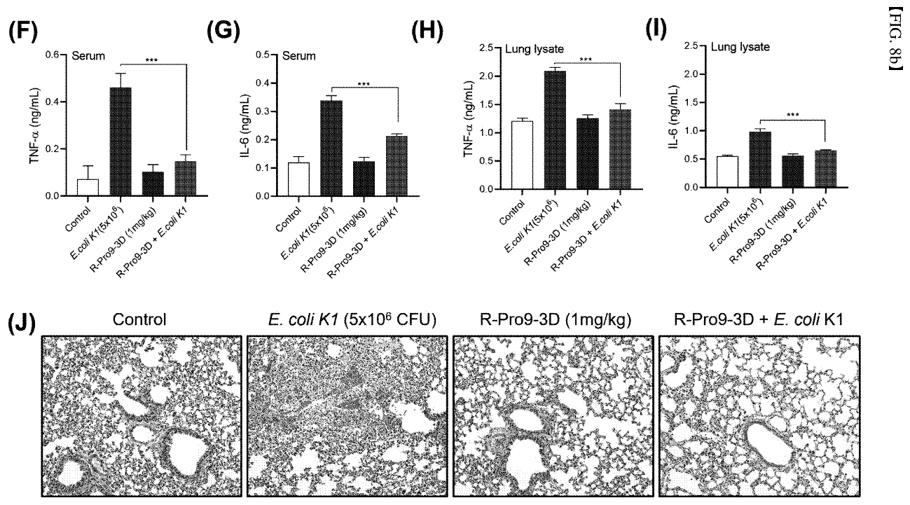

In a specific examplary embodiment of the present invention, the efficacy of R-Pro9-3D as a peptide antibiotic was confirmed in a septic shock mouse model established using E. coli K1. As shown in FIG. 8A, it was confirmed that R-Pro9-3D reduced the number of bacteria in the lungs, liver, and kidneys of mice. These results indicate that R-Pro9-3D effectively suppresses bacterial growth in the visceral organs of mice infected with E. coli K1.

Further, as shown in FIG. 8B, as a result of conducting serum endotoxin measurements, it was confirmed that pretreatment with R-Pro9-3D effectively reduced endotoxin levels.

In addition, as shown in FIGS. 8C to 8E, it was confirmed that pretreatment with R-Pro9-3D remarkably reduced AST, ALT, and BUN levels in mice infected with E. coli K1.

Furthermore, as shown in FIGS. 8F to 8I, it was confirmed that pretreatment with R-Pro9-3D remarkably reduced the levels of TNF-α and IL-6 cytokines in the serum or lungs of mice infected with E. coli K1.

Further, as shown in FIG. 8J, the histological pattern of lung tissue infected with E. coli K1 showed severe edema, pulmonary congestion, and alveolar hemorrhage with PMNs infiltrated into the lungs. Such microanatomical features imply that increased PMNs are associated with lung injury to induce severe sepsis during infections with E. coli K1. In contrast, as a result of confirming the lung tissues of septic mice pretreated with R-Pro9-3D, the therapeutic effect on sepsis was confirmed by confirming that the pathological changes induced by E. coli K1 were effectively alleviated by R-Pro9-3D.

That is, since it was confirmed in the present invention that the R-Pro9-3D peptide can act as a new peptide antibiotic to prevent sepsis-related inflammation in mice infected with E. coli K1, R-Pro9-3D of the present invention may be usefully applied as a new peptide antibiotic for the effective treatment of sepsis or septic shock.

In yet another aspect, the present invention relates to a quasi-drug composition for preventing or ameliorating Gram-negative bacterial infectious diseases, including the peptide as an active ingredient.

In the present invention, the Gram-negative bacterial infectious disease may be characterized by being Gram-negative sepsis or septic shock, but is not limited thereto.

In yet another aspect, the present invention relates to a food composition for preventing or ameliorating Gram-negative bacterial infectious diseases, including the peptide as an active ingredient.

In the present invention, the Gram-negative bacterial infectious disease may be characterized by being Gram-negative sepsis or septic shock, but is not limited thereto.

In yet another aspect, the present invention relates to a method for preventing or treating Gram-negative bacterial infectious diseases, the method including: administering a pharmaceutical composition including the peptide as an active ingredient to a patient with a Gram-negative bacterial infectious disease.

In the present invention, the Gram-negative bacterial infectious disease may be characterized by being Gram-negative sepsis or septic shock.

In yet another aspect, the present invention provides a use of the peptide for preventing or treating Gram-negative bacterial infectious diseases.

In the present invention, the Gram-negative bacterial infectious disease may be characterized by being Gram-negative sepsis or septic shock, but is not limited thereto.

In yet another aspect, the present invention provides a use of the peptide for preparing a drug for preventing or treating Gram-negative bacterial infectious diseases.

In the present invention, the Gram-negative bacterial infectious disease may be characterized by being Gram-negative sepsis or septic shock, but is not limited thereto.

As used herein, the term “peptide” refers to a linear molecule formed by bonding amino acid residues to each other by peptide bonds. The peptide of the present invention may be prepared by a chemical synthesis method known in the art, particularly, solid-phase synthesis techniques (Merrifield, J. Amer. Chem. Soc. 85:2149-54(1963); Stewart, et al., Solid Phase Peptide Synthesis, 2nd.ed. ed., Pierce Chem. Co.: Rockford, 111(1984)), and may also be produced by genetic engineering techniques.

Specifically, the peptide of the invention may be prepared by a standard synthetic method, a recombinant expression system, or any other methods in the art. Therefore, the peptides of the invention may be synthesized, for example, by a number of methods, including:
(a) a method of synthesizing the peptides stepwise or by fragment assembly by means of solid phase or liquid phase methods and isolating and purifying a final peptide product; or
(b) a method of expressing a nucleic acid construct encoding a peptide in a host cell and
recovering an expression product from a host cell culture; or
(c) a method for performing cell-free in vitro expression of a nucleic acid construct encoding a peptide and recovering an expression product; or
a method of obtaining the fragments of a peptide using any combination of (a), (b), and (c), then ligating the fragments to obtain a peptide, and recovering the peptide.

As used herein, the term “prevention” refers to all actions that suppress or delay the occurrence, spread, and recurrence of Gram-negative bacterial infectious diseases by administering the protein according to the present invention or fragments thereof, or a composition including the same.

As used herein, term “treatment” refers to all actions that ameliorate or beneficially change the symptoms of Gram-negative bacterial infectious diseases and complications thereof by administering the protein according to the present invention or fragments thereof, or a composition including the same. A person with ordinary skill in the art to which the present invention pertains should be able to refer to the data presented by the Korean Medical Association, and the like to understand the accurate criteria for diseases for which the composition of the present invention is effective and judge the degree of alleviation, amelioration and treatment.

A therapeutically effective amount of the composition of the present invention may vary depending on various factors, such as the method of administration, the intended site, and the condition of a patient. Therefore, when the composition of the present invention is used in the human body, the dosage should be determined appropriately in consideration of both safety and efficiency. It is also possible to estimate an amount used in humans from an effective amount determined through animal experiments. These considerations in determining an effective amount are described, for example, in Hardman and Limbird, eds., Goodman and Gilman's The Pharmacological Basis of Therapeutics, 10th ed.(2001), Pergamon Press; and E. W. Martin ed., Remington's Pharmaceutical Sciences, 18th ed.(1990), Mack Publishing Co.

The pharmaceutical composition of the present invention is administered in a pharmaceutically effective amount. As used herein, the term “pharmaceutically effective amount” refers to an amount that is sufficient to treat diseases at a reasonable benefit/risk ratio applicable to medical treatment and does not cause side effects, and an effective dose level may be determined according to factors including patient's health status, type of diseases, severity of disease, activity of drugs, sensitivity to drugs, administration method, administration time, administration route and excretion rate, duration of treatment, and drugs used in combination or simultaneously, and other factors well known in the medical field. The composition of the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by a person skilled in the art.

The pharmaceutical composition of the present invention may include a carrier, a diluent, an excipient, or a combination of two or more thereof, which are typically used in biological preparations. As used herein, the term “pharmaceutically acceptable” refers to exhibiting non-toxic characteristics to cells or humans exposed to the composition. The carrier is not particularly limited as long as it is suitable for in vivo delivery of the composition, and for example, compounds described in Merck Index, 13th ed., Merck & Co. Inc., saline, sterile water, Ringer's solution, buffered saline, a dextrose solution, a maltodextrin solution, glycerol, ethanol, and one or more of these components may be mixed and used, and other conventional additives such as an antioxidant, a buffer, and a bacteriostat may be added, if necessary. Further, the pharmaceutical composition of the present invention may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition of the present invention may be preferably prepared according to each disease or ingredient using as a suitable method in the art or a method disclosed in Remington's Pharmaceutical Science (Mack Publishing Company, Easton PA, 18th, 1990).

In an exemplary embodiment, the pharmaceutical composition may be one or more formulations selected from the group including an oral formulation, an external preparation, a suppository, a sterile injectable solution, and a spray.

The composition of the present invention may also include a carrier, a diluent, an excipient, or a combination of two or more thereof, which are typically used in biological preparations. A pharmaceutically acceptable carrier is not particularly limited as long as it is suitable for in vivo delivery of the composition, and for example, compounds described in Merck Index, 13th ed., Merck & Co. Inc., saline, sterile water, Ringer's solution, buffered saline, a dextrose solution, a maltodextrin solution, glycerol, ethanol, and one or more of these components may be mixed and used, and other conventional additives such as an antioxidant, a buffer, and a bacteriostat may be added, if necessary.

Further, the composition of the present invention may be prepared in injectable formulations such as an aqueous solution, a suspension, and an emulsion, pills, capsules, granules, or tablets by further adding a diluent, a dispersant, a surfactant, a binder, and a lubricant. Furthermore, the composition of the present invention may be preferably prepared according to each disease or ingredient using as a suitable method in the art or a method disclosed in Remington's Pharmaceutical Science (Mack Publishing Company, Easton PA, 18th, 1990).

The composition of the present invention may additionally contain one or more active ingredients that exhibit the same or similar functions. The composition of the present invention contains 0.0001 to 10 wt %, preferably 0.001 to 1 wt % of the protein, based on the total weight of the composition.

The pharmaceutical composition of the present invention may further include a pharmaceutically acceptable additive, and in this case, as the pharmaceutically acceptable additive, it is possible to use starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropylcellulose, opadry, sodium starch glycolate, carnauba lead, synthetic aluminum silicate, stearic acid, magnesium stearate, aluminum stearate, calcium stearate, white sugar, dextrose, sorbitol, talc, and the like. It is preferred that the pharmaceutically acceptable additive according to the present invention is included in an amount of 0.1 parts by weight to 90 parts by weight based on the composition, but the amount is not limited thereto.

The composition of the present invention may be administered parenterally (for example, intravenously, subcutaneously, intraperitoneally, or locally) or orally depending on the desired method, and the dosage range may vary depending on the body weight, age, sex, and health condition of a patient, a diet, an administration time, an administration method, an excretion rate, and the severity of a disease. The daily dosage of the composition according to the present invention is 0.0001 to 10 mg/ml, preferably 0.0001 to 5 mg/ml, and more preferably once a day or divided into several doses.

A liquid preparation for the oral administration of the composition of the present invention corresponds to a suspension, a liquid for internal use, an emulsion, a syrup, and the like, and the liquid preparation may together include various excipients, for example, a wetting agent, a sweetener, an aroma, a preservative, and the like, in addition to water and liquid paraffin which are typically used simple diluents. Examples of a preparation for parenteral administration include an aqueous sterile solution, a non-aqueous solvent, a suspension, an emulsion, a freeze-dried preparation, a suppository, and the like.

The quasi-drug composition of the present invention refers to products which have a milder action than a drug among products used for the purpose of diagnosing, treating, ameliorating, alleviating, treating, or preventing a human or animal disease, and for example, according to the Pharmaceutical Affairs Act, the quasi-drug is a product excluding those used for pharmaceutical purposes, and includes products used to treat or prevent diseases in humans or animals, products that have little or no direct effect on the human body, and the like.

The quasi-drug composition of the present invention may be a transdermal formulation such as a lotion, an ointment, a gel, a cream, a patch, or a spray. In each formulation, the quasi-drug composition may be blended with other components selected arbitrarily depending on the formulation or purpose of use of other quasi-drugs, and the like. The mixing amount of the active ingredient may be suitably determined depending on the purpose of use (for suppression or alleviation). For example, the quasi-drug composition of the present invention may include conventional adjuvants such as thickeners, stabilizers, solubilizing agents, vitamins, pigments and fragrances, carriers, and the like. In addition, in the quasi-drug composition of each formulation, components other than the above-mentioned essential components may be selected and blended by those skilled in the art without difficulty depending on the formulation, purpose of use, and the like.

The food composition of the present invention may be a compound of the present invention or a pharmaceutically acceptable salt thereof itself, or a composition mixed with a sitologically acceptable carrier. In this case, the content of the compound of the present invention or a pharmaceutically acceptable salt thereof may be appropriately adjusted by a conventional method based on the content and dosage in the pharmaceutical composition.

The most desirable aspect of the food composition of the present invention is a health functional food composition, but is not limited thereto, and other aspects include general food forms such as processed meat products, fish products, tofu, jelly, rice porridge, noodles such as ramen or rice noodles, seasonings such as soy sauce, soybean paste, red pepper paste, and mixed paste, sauces, sweets, dairy products such as fermented milk or cheese, pickled foods such as kimchi or pickles, fruits and vegetables, and beverages such as soy milk, and fermented drinks. As another aspect, the food composition of the present invention may be a food additive.

Meanwhile, the pharmaceutically acceptable carrier may also be used as a sitologically acceptable salt.

### Statistical Analysis

All data was analyzed by the Kaplan-Meier log rank test and one-way and two-way ANOVA, and then statistically analyzed by Dunnett's test using GraphPad Prism (GraphPad Software Inc., La Jolla, CA, USA). All experimental data was expressed as the mean±SEM of three or more replicates. Values indicate statistically significant at *P<0.05, **P<0.01 and ***P<0.001, and ns indicates non-significant values.

These examples are only for exemplifying the present invention, and it will be apparent to those of ordinary skill in the art that the scope of the present invention should not be construed as being limited by these examples.

### [Example 1] Bacterial Strains Used in Experiment

Among the bacterial strains used in the experiment, Escherichia coli KCTC 1682 was obtained from the Korean Collection for Type Cultures (KCTC) (Jeongeup, Korea), Acinetobacter baumannii KCCM 40203 and Pseudomonas aeruginosa KCCM 11328 were obtained from the Korean Culture Center of Microorganisms (KCCM) (Seoul, Korea), and Klebsiella pneumonia NCCP 16054 was obtained from the National Culture Collection for Pathogens (NCCP) (Osong, Korea). In addition, carbapenem-resistant E. coli NCCP16044 and carbapenem-resistant Klebsiella pneumonia NCCP 15782 were obtained from the NCCP, and three types of carbapenem-resistant A. baumanii CCARM 12004-6 were obtained from the Culture Collection of Antimicrobial Resistance Microbes (CCARM) (Cheongju City, Korea). The E. coli K1 (ATCC700973) strain used in the animal experiments was obtained from the American Type Culture Collection (ATCC) (Rock Valley, USA).

### [Example 2] Design and Synthesis of Peptides

The inventors of the present invention designed and synthesized Pro9-3 from the active site of protaetiamycine, a natural antibiotic peptide with 43 amino acid residues, as an insect defensin in a previous study. However, despite the potent antimicrobial activity thereof, Pro9-3 and its enantiomeric peptide Pro9-3D exhibit severe cytotoxicity to mammalian cells (Table 1).

In the present invention, the present inventors designed a peptide sequence for alleviating the severe cytotoxicity of Pro9-3 and Pro9-3D while maintaining a short length of 9 amino acids to secure the same cationic, hydrophobic and antimicrobial activities as Pro9-3 and its enantiomer peptide Pro9-3D. The present inventors designed R-Pro9-3 and R-Pro9-3D in which the peptide sequences of Pro9-3 and Pro9-3D were reversed to have two sequential Arg residues at the N-terminus, increasing the cationic properties of the N-terminus (Table 1). Furthermore, the present inventors designed an enantiomer R-Pro9-3D by replacing the L-amino acids of R-Pro9-3 with D-amino acids. The helical wheel projections of all the peptides exhibits highly cationic amphipathic properties (FIG. 1), indicating that the peptides form an amphipathic alpha helical structure in the bacterial membrane and can effectively penetrate the bacterial cell membrane.

TABLE 1
Synthesized peptides and physicochemical properties thereof

| | | | | | Hydrophobic | | |
|---|---|---|---|---|---|---|---|
| | | | Molecular | | moment | Hydrophobicity | SEQ |
| Peptides | Sequence^{a} | Length | weight | Charge | <μH>^{b} | <H>^{b} | ID NO: |
| R-Pro9-3 | RRWIALWLR- | 9 | 1269 | +3 | 0.692 | 0.776 | 1 |
| | NH2 | | | | | | |
| R-Pro9-3D | rrwialwlr-NH2 | 9 | 1269 | +3 | 0.692 | 0.776 | 2 |
| Pro9-3 | RLWLAIWRR- | 9 | 1269 | +3 | 0.692 | 0.776 | 3 |
| | NH2 | | | | | | |
| Pro9-3D | rlwlaiwrr-NH2 | 9 | 1269 | +3 | 0.692 | 0.776 | 4 |

^{a}In Table 1, lowercase letters in the sequence indicate D-amino acids.
^{b}In Table 1, the hydrophobic moment <μH> and hydrophobicity <H> were calculated by http://heliquest.ipmc.cnrs.fr/cgi-bin/ComputParams.py.

All the peptides were synthesized by solid-phase synthesis methods using N-(9-fluorenyl)methoxycarbonyl amino acids, and the molecular weight of the peptide was confirmed using a matrix-assisted laser desorption ionization time-of-flight (MALDI-TOF) mass spectrometer.

### [Example 3] Measurement of Antimicrobial Activities of Peptides

The antimicrobial activities of the peptides were measured by a minimal inhibitory concentration (MIC) analysis. The MIC was determined using a broth dilution method. All bacterial strains were grown overnight at 37° C. in a Muller-Hinton (MH) medium. After peptides (Pro9-3, Pro9-3D, R-Pro9-3, and R-Pro9-3D) were added to bacterial suspensions (2×10⁵ CFU/mL, MH medium) using existing antibiotics (imipenem, meropenem) and melittin as controls and cultured for 16 hours, bacterial growth was read at 600 nm using a SpectraMAX microplate reader (Molecular Devices, San Jose, USA).

The antimicrobial activities of Pro9-3, Pro9-3D, R-Pro9-3, R-Pro9-3D and controls (imipenem, meropenem, and melittin) against standard Gram-negative bacteria and carbapenem-resistant E. coli (CREC), A. baumannii (CRAB) and K. pneumoniae (CRKP) were investigated. Compared to the melittin control and the existing antibiotics, R-Pro9-3 consisting of L-type amino acids showed a bactericidal effect similar to that of the parent peptide Pro9-3. In contrast, their enantiomeric peptides, R-Pro9-3D and Pro9-3D, showed much better antimicrobial activity against Gram-negative bacterial strains than L-type peptides (Table 2).

Next, the effects of the peptides (Pro9-3, Pro9-3D, R-Pro9-3, and R-Pro9-3D) against carbapenem-resistant bacterial strains were evaluated. It was confirmed that among all the peptides including melittin, R-Pro9-3D exhibits the most potent activity against CREC, CRAB, and CRKP carbafenem-resistant bacterial strains. R-Pro9-3D has the most potent activity against, particularly, CRAB. Further, it was confirmed that R-Pro9-3D showed the most potent antibacterial activity against all Gram-negative bacteria, including standard bacteria and resistant bacteria, in the order of geometric mean (GM) values (Table 2): R-Pro9-3D(5.8)<Pro9-3D(7.6)<melittin (12.4)<R-Pro9-3(24.9)<Pro9-3(32.0). In addition, R-Pro9-3D (34.6) also had the highest relative selective index, and the relative selective index was higher in the order of Pro9-3D (26.5), Pro9-3D (8.0), and Pro9-3 (6.3). Melittin (0.25), which is the control, had excellent antimicrobial activity, but had a very low relative selective index value due to its high hemolytic activity. Since CREC, CRAB and CRKP strains are already resistant to carbapenem antibiotics such as imipenem and meropenem, such antibiotics did not show antimicrobial activity against carbapenem-resistant strains.

TABLE 2
Comparison of antimicrobial activity of peptide and control against standard
Gram-negative bacteria and carbapenem-resistant Gram-negative bacteria

| | Minimal inhibitory concentration (MIC) in μM | | | | | | |
|---|---|---|---|---|---|---|---|
| Microorganisms | Pro9-3 | Pro9-3D | R-Pro9-3 | R-Pro9-3D | Melittin | Imipenem | Meropenem |

Standard Gram-negative bacteria

| | | | | | | | |
|---|---|---|---|---|---|---|---|
| E. coli | 16 | 8 | 16 | 8 | 8 | 0.5 | 0.25 |
| A. baumannii | 16 | 4 | 16 | 4 | 4 | 1 | 0.5 |
| P. aeruginosa | 64 | 8 | 64 | 8 | 16 | 1 | 1 |
| K. pneumonia | 64 | 8 | 32 | 8 | 32 | <0.25 | <0.25 |

Carbapenem-resistant Gram-negative bacteria

| | | | | | | | |
|---|---|---|---|---|---|---|---|
| CREC 16044 | 16 | 8 | 16 | 4 | 8 | 8 | 8 |
| CRAB 12004 | 16 | 8 | 32 | 4 | 4 | 64 | 128 |
| CRAB 12005 | 16 | 8 | 16 | 4 | 4 | 64 | 128 |
| CRAB 12006 | 16 | 8 | 16 | 4 | 4 | 64 | 128 |
| CRKP 15782 | 64 | 8 | 16 | 8 | 32 | >128 | >128 |
| ^{a}GM | 32 | 7.6 | 24.9 | 5.8 | 12.4 | NA | NA |
| ^{b}HC₁₀ | 200 | 200 | 200 | 200 | 3.1 | NA | NA |
| ^{c}Relative | 6.3 | 26.5 | 8.0 | 34.6 | 0.25 | NA | NA |
| selective index | | | | | | | |

^{a}Geometric means (GMs) are the mean minimum inhibitory concentration (MIC) values for all bacterial strains
^{b}HC10 indicates the peptide concentration that induces 10% hemolysis of sheep heparinized red blood cells in vitro.
^{c}Relative selective index is the minimum peptide concentration that causes 10% hemolysis. When no detectable hemolysis was observed at 100 μM, a value of 200 μM was used to calculate the selectivity index, and the selectivity index was calculated by the GM formula of HC10/MIC (μM). Higher values mean greater cell selectivity for bacteria, and NA indicates not applicable.

### [Example 4] Mechanism of Antibacterial Activity of Peptides

In the present invention, the antibacterial mechanism of peptides was investigated. LPS is a major component of the outer membrane of Gram-negative bacteria and induces TLR4-mediated inflammatory signaling. There is growing attention towards treating Gram-negative infections using peptides with strong anti-endotoxin activity which can eliminate bacterial LPS.

The binding ability of peptides to LPS was investigated by BODIPY-TR cadaverine (BC, Thermo Fisher Scientific, MA, USA) displacement assay (FIG. 2A). A probe complex was prepared by mixing 5 μg/mL BC and 5 μg/mL LPS (E. coli O111:B4, Sigma-Aldrich, St. Louis, MO, USA) in a 50 mM Tris buffer (pH 7.4) at room temperature for 6 hours. Peptides containing a polymyxin B (PMB) control (1 to 64 μM), which is known to have high LPS binding ability, were allowed to interact with the LPS-BC mixture in a black 96-well plate for 30 minutes. Relative fluorescence intensities were recorded using a fluorescence microplate reader (Molecular Devices, San Jose, USA) at an excitation wavelength of 580 nm and an emission wavelength of 620 nm.

As a result, compared to the well-known LPS-binding peptide PMB (76.6%), Pro9-3, Pro9-3D, R-Pro9-3 and R-Pro9-3D (4 μM) exhibited excellent LPS binding ability while increasing BC displacement by 42.0, 51.2, 40.6 and 51.3%, respectively (FIG. 2A).

Peptide-mediated LPS-neutralizing activity was determined using a Limulus amebocyte lysate (LAL) chromogenic endotoxin quantification kit (GenScript, New Jersey, USA). 3.1, 6.3, 10, 12.5, 25, and 50 μM of each peptide were allowed to react so as to interact with LPS (2 ng/mL) at 37° C. for 10 minutes. LL-37, which is known to have high LPS-neutralizing ability, was used as a control. After an LAL enzyme (10 μL) was added to the peptide-LPS complex and the resulting mixture was allowed to react for 10 minutes, a chromogenic substrate was added thereto. An endotoxin level was shown by measuring the absorbance at 545 nm in triplicate and expressed in endotoxin units (EUs) per milliliter.

As a result, all the peptides neutralized LPS in a concentration-dependent manner: R-Pro9-3D, 51.6%; Pro9-3D, 27.9%; Pro9-3, 11.3%; R-Pro9-3, 17.3%; LL-37, 76.5% (FIG. 2B). Therefore, it was confirmed that R-Pro9-3D has better LPS neutralization ability than Pro9-3 and Pro9-3D.

Depolarization ability by peptides was measured using a membrane potential-sensitive dye 3,3′-dipropylthiadicarbocyanine iodide (diSC₃-5) using intact CRAB bacteria. CRAB cells were washed using a wash buffer (5 mM HEPES, 20 mM glucose, pH 7.4). The cells were resuspended in a dilution buffer (5 mM HEPES, 20 mM glucose, 0.1 M KCl, pH 7.4) and then cultured with a diSC₃-5 dye (1 hour). Finally, the fluorescence changes of cells treated with various concentrations of peptides, a negative control (cells with dye) and a positive control (1% Triton X-100) were recorded using a fluorescence spectrophotometer, and the results are expressed as percent depolarization.

As a result, all the peptides increased diSC₃-5 fluorescence in a concentration-dependent manner in a range similar to that of melittin (FIG. 2C). In particular, 4 μM Pro9-3, Pro9-3D, R-Pro9-3, R-Pro9-3D, and melittin increased depolarization by 66.3, 68.4, 66.7, 67.8, and 75.6%, respectively, and these peptides were shown to target CRAB membranes.

Thus, the above results suggest that the antibacterial mechanism of these peptides is associated with the strong interaction of peptides and LPS in the bacterial cell membrane and cell membrane permeability.

### [Example 5] Effect of Peptides on Biofilm Inhibition

The biofilm formation of carbapenem-resistant Gram-negative bacteria increases antibiotic resistance as a self-defense mechanism to survive in an adverse environment. Microbial interactions with surface materials mediate primary adhesion and development as a biofilm structure surrounded by a matrix.

A crystal-violet-based assay was used to investigate the biofilm inhibition ability of R-Pro9-3D against A. baumannii standard bacteria and a CRAB 12006 line. Bacterial cells (2×10⁵ CFU/mL in an MH medium containing 0.2% (w/v) glucose) were exposed to various concentrations (0-32 μM) of the peptides, melittin, imipenem, and meropenem at 37° C. for 16 hours. Thereafter, bacterial cells were fixed with methanol (100%, 15 minutes) and then stained with crystal violet (0.1% (w/v) in 0.25% (v/v) acetic acid for 1 hour). The plate was washed with distilled H₂O, dried and dissolved in ethanol (100% v/v). Color development, which indicates the extent of biofilm, was measured at 595 nm and expressed as biofilm formation percentage.

As a result, treatment with a control antibiotic containing all the Pro9-3 peptides and melittin killed biofilm-forming A. baumannii and CRAB 12006 for 16 hours (FIG. 3). In particular, peptide R-Pro9-3D was excellent in killing biofilm-forming CRAB 12006, as demonstrated by crystal violet absorbance being sharply reduced by 96.1% at MIC, when compared to the other peptides. Due to acquired resistance, the ability of imipenem and meropenem to inhibit the biofilm formation of CRAB 12006 was remarkably lower than that of the A. baumannii standard strain. As such, it was confirmed that R-Pro9-3D is particularly effective in removing pre-formed biofilms due to its strong membrane permeability and inhibiting CRAB biofilms by translocating the biofilm matrix.

### [Example 6] Resistance of Peptide to Protease

Proteolysis of peptides in serum has limited clinical use due to decreased bioavailability and half-life. Therefore, the stability of all the peptides pre-exposed to trypsin and chymotrypsin proteases was investigated and the relative growth inhibition of E. coli and A. baumannii was determined (Table 3). All the peptides at the MIC were pre-cultured with proteolytic enzymes such as trypsin and α-chymotrypsin (ratio of 10,000:1; peptide:enzyme) at 37° C. for 6 hours. Thereafter, 100 μL of a peptide-protease mixture was added to 100 μL of a bacterial suspension (E. coli, A. baumannii, 2×10⁵ CFU/mL), and the resulting mixture was further cultured at 37°° C. for 16 hours. A bacterial suspension without a peptide and protease was employed as a negative control. An analysis was performed in triplicates through independent experiments, and the degree of bacterial growth was measured at 600 nm using a microplate reader.

As a result, it was found that Pro9-3 and R-Pro9-3 did not affect the survival of bacterial strains (E. coli and A. baumannii) even at 64 μM, whereas their enantiomers Pro9-3D and R-Pro9-3D effectively maintained bactericidal efficacy without affecting the MIC (Table 3). This means that the proteolytic stability is higher due to D-amino acid substitution.

TABLE 3
Inhibition of antibacterial activity of Pro9-3 and its analogs by
trypsin and α-chymotrypsin against E. coli and A. baumannii.

| | Minimal inhibitory concentration (MIC) in μM | | | |
|---|---|---|---|---|
| Microorganisms | Pro9-3 | Pro9-3D | R-Pro9-3 | R-Pro9-3D |
| E. coli + Trypsin | >64 | 8 | >64 | 8 |
| E. coli + α-Chymotrypsin | >64 | 8 | 64 | 8 |
| A. baumannii + Trypsin | >64 | 4 | >64 | 4 |
| A. baumannii + α-Chymotrypsin | >64 | 4 | >64 | 4 |

### [Example 7] Comparison of Secondary Structures of Peptides

The secondary structural changes of all the peptides in aqueous solution and membrane mimicking environment were examined by CD measurements. All CD experiments on the peptides were performed using a J-810 spectropolarimeter (Jasco, Tokyo, Japan) with a 1 mm path length cell at 25° C. The CD spectra of the peptides were recorded at 0.1 nm intervals from 190 to 250 nm at 100 μM. The CD experiments were performed in an aqueous solution or 50 mM MDPC micelles (membrane-mimicking environment). The average value of the CD spectral data obtained through 10 scans was derived and smoothed using J-810. CD data was expressed as mean residue ellipticity (θ) in units of deg cm² dmol⁻¹.

As a result, all the peptides showed a random coil structure in an aqueous environment (FIG. 4A) but showed a fully helical structure in the presence of dodecylphosphocholine (DPC) micelles (FIG. 4A). In particular, the CD spectra of all the peptides in the DPC micelles clearly showed double negative maxima or minima at 205 nm and 220 nm (FIG. 4B). The double negative maxima at 205 nm and 220 nm are typical characteristics of α-helical structures of peptides. Pro9-3D has the opposite shape of L-peptides, which is a common occurrence for distinct amino acid conformations. Since Pro9-3D and its reverse sequence (R-Pro9-3D) exhibit the same chirality, their CD spectra represents the mirror images of the parent peptide. In addition, R-Pro9-3D has a higher content of alpha helical structure than Pro9-3D in DPC micelles (FIG. 4B). This means that R-Pro9-3D has higher contents of α-helical structure compared to Pro9-3D.

### [Example 8] Cytotoxicity Analysis of Peptides

The toxic effects of R-Pro9-3 and R-Pro9-3D on different mammalian cells were investigated and compared to Pro9-3 and Pro9-3D (FIG. 5). Peptide-induced toxicity was determined by hemolysis of sheep red blood cells (sRBCs). Fresh sRBCs were washed three times or more with phosphate buffered saline (PBS), and debris was removed by centrifugation (1000×g, 5 minutes, 4° C.). All the peptides including a melittin control (0.2 to 100 μM) and a positive control (1% (v/v) Triton X-100) were cultured with 4% (v/v) sRBCs at 37° C. for 1 h, and then centrifuged at 1000×g for 5 minutes. The supernatant was measured at 405 nm using a microplate reader.

As a result, R-Pro9-3, R-Pro9-3D, Pro9-3, and Pro9-3D induced very low hemolytic activity (3.4%, 1.2%, 2.5%, and 1.9%, respectively) in sheep red blood cells (sRBCs) even at 100 μM, whereas melittin, a well-known AMP with broad-spectrum antimicrobial activity and high cytotoxicity showed 100% hemolysis at 25 μM (FIG. 5A). R-Pro9-3D had the highest relative selective index (34.6) against the Gram-negative bacteria tested, and the relative selective indices of Pro9-3D, Pro9-3, and R-Pro9-3 were 26.5, 6.3, and 8.0, respectively (Table 2).

Additionally, for a cell-based toxicity assay, RAW 264.7 mouse macrophages and human kidney (HK)-2 cells were obtained from the Korean Cell Line Bank (Seoul, Korea), and cells were maintained in in a DMEM culture medium (Thermo Fischer Scientific Inc., MA, USA) supplemented with 10% fetal bovine serum and 1% penicillin/streptomycin at 37° C. in a humidified 5% CO₂ incubator. The cytotoxicity of the peptides was analyzed using a WST-8 cell proliferation assay (Biomax Co, Ltd, Seoul, South Korea), and the experiment was performed according to the kit protocol. Cells (1×10⁴) were seeded in a 96-well plate, the peptide treatment (0 to 100 μM) were initiated at 80% confluency and then the cells were cultured for 24 hours. After culturing, a WST-8 reagent was added and the change in absorbance was read at 450 nm relative to the reagent blank, and values were expressed as percent cell survival.

As a result of cytotoxicity analysis, it was found that neither R-Pro9-3 nor Pro9-3 showed toxicity compared to melittin even at a concentration up to 100 μM (FIGS. 5B and 5C). In contrast, Pro9-3D exhibited a toxicity of 44.5% and 22.9%, respectively, in RAW264.7 and human kidney (HK)-2 cells at a concentration of 100 μM, and R-Pro9-3D exhibited a low toxicity of 20.3% and 9.6%. These results indicate that, particularly, R-Pro9-3D exhibited greater bacterial cell selectivity and less cytotoxicity than the other peptides.

### [Example 9] Confirmation of Anti-Inflammatory Activity of Peptides

Nitric oxide (NO) overproduction is known to be associated with acute and/or chronic inflammation in response to bacterial lipopolysaccharide (LPS). Since R-Pro9-3D effectively interacts with LPS and exhibits antimicrobial activity, it was hypothesized that R-Pro9-3D could regulate LPS-induced inflammatory signals. The inhibitory effect of the peptides on nitrite production was evaluated by Griess analysis. RAW264.7 cells (1×10⁵) were pretreated with various concentrations (1, 5, 25, and 50 μM) of peptide for 1 hour and then stimulated with 20 ng/mL LPS for 16 hours. After culturing, the supernatant and Griess reagent were added at the same ratio. Color changes were measured at 540 nm. The concentration of nitrite content was evaluated using a standard curve of sodium nitrite. In addition, the production of inflammatory cytokines including TNF-α and IL-6 in a culture medium was quantified using an enzyme-linked immunosorbent assay kit (ELISA; R&D Systems, Minneapolis, MN, USA) and analyzed according to the kit protocol. All analyses were performed three times, and the concentrations of TNF-α and IL-6 were measured by measuring the absorbance at 450 nm using a microplate reader.

As a result, all the peptides significantly suppressed NO secretion in a dose-dependent manner compared to melittin (FIG. 6). In particular, R-Pro9-3D showed the greatest inhibitory effect compared to R-Pro9-3, Pro9-3D and Pro9-3 (47% at 25 μM and 81% at 50 μM). It was confirmed through the ELISA-based analysis of tumor necrosis factor-α (TNF-α) and interleukin-6 (IL-6) levels that the release of cytokines was significantly increased in LPS-RAW264.7 cells (FIGS. 6B and 6C). R-Pro9-3D at a concentration of 25 μM suppressed TNF-α by 52.1% and IL-6 by 47.2%, whereas Pro9-3, Pro9-3D and R-Pro9-3 suppressed TNF-α and IL-6 to a relatively lesser extent. That is, by confirming the ability of the peptides to suppress nitrite and inflammatory cytokines, the excellent anti-inflammatory effect of R-Pro9-3D of the present invention was confirmed.

Through the above results, it was confirmed that R-Pro9-3D of the present invention has much better antimicrobial properties, LPS binding, antibiofilm properties, and proteolysis resistance than the other peptides, and has a potent anti-inflammatory effect.

### [Example 10] Confirmation of Anti-Septic Activity of Peptides in E.coli K1-Induced Septic Shock Mouse Model

Based on the antimicrobial, antibiofilm and anti-inflammatory effects in LPS-RAW 264.7 cells, the anti-septic effect of R-Pro10-1D was additionally analyzed in an E. coli K1-induced septic shock mouse model.

Female ICR mice were purchased from Orient (Daejeon, Korea) and housed in a specific pathogen-free (SPF) and humidity controlled environment. All procedures were reviewed and approved by the Institutional Animal Care and Use Committee (IACUC) of Konkuk University, Korea (IACUC No.: KU20192, Apr. 5, 2021).

For in vivo toxicity measurements, ICR mice (n=5 per group) were injected intraperitoneally (i.p.) with R-Pro9-3D (1 mg and 5 mg/kg/day in PBS), and serum levels of aspartate aminotransferase (AST) and alanine aminotransferase (ALT) in the liver and BUN (blood urea nitrogen) in the kidney were measured using a standard kit manufactured by ASAN PHARM. Co., LTD. It was confirmed that R-Pro9-3D did not increase the above enzyme levels (FIG. 7), and a higher dose of R-Pro9-3D is also non-toxic.

Twenty ICR mice were randomly divided into four groups (5 mice per group), and mice injected intraperitoneally (i.p.) with PBS were used as a normal control. The peptide control group mice were injected intraperitoneally (i.p.) with R-Pro0-3D (1 mg/kg). The bacterial control was injected with E. coli K1 (5×10⁶ CFU/mouse). Mice injected with E. coli K1 and R-Pro9-3D were injected with R-Pro9-3D 1 hour before injecting E. coli K1, and 16 hours later, the mice were euthanized and the lungs, liver, and kidneys were aseptically removed, and then homogenized using ice-cold PBS. To assess the relative abundance of E. coli K1, all homogenates (1:1000, PBS) were plated on Luria-Bertani agar and the number of bacteria colonies was measured. The levels of inflammatory cytokines (TNF-α and IL-6) were measured in serum and lung lysates using an ELISA kit (R&D Systems, Minneapolis, MN, USA). The contents of AST, ALT, and BUN were measured using a standard kit manufactured by ASAN PHARM. Co., LTD.

As a result, in a sepsis model, mice injected with E. coli K1 (5×10⁶ CFU/mouse) had increased bacterial loads in vital organs such as lungs, liver, and kidneys, leading to severe organ damage (FIG. 8A). Furthermore, E. coli K1 rapidly released excessive levels of endotoxins into the circulatory system (FIG. 8B) and increased the serum levels of organ damage markers (AST, ALT and BUN) and the production of inflammatory cytokines (FIGS. 8C to 8I). In contrast, it was confirmed that the pretreatment with 1 mg/kg R-Pro9-3D significantly inhibited bacterial growth, and bacterial growth was also effectively inhibited in the lysates of the vital organs (FIG. 8A). In addition, R-Pro9-3D pretreatment reduced endotoxin levels by 55.4% in E. coli K1-injected mice (FIG. 8B), and reduced AST, ALT, and BUN levels by 29.3%, 25.9%, and 19.8%, respectively, compared to the bacterial control (FIGS. 8C to 8E). Furthermore, the ability of R-Pro9-3D to regulate a cytokine storm was investigated, and it was confirmed that TNF-α and IL-6 levels were downregulated in serum and lung samples compared to E. coli K1-infected samples (FIGS. 8F to 8I).

Further, to determine the microanatomical characteristics of polymorphism-neutrophil infiltrates, 5-μm thick sections were prepared from paraffin-blocked lungs and sequentially processed for hematoxy and eosin (H&E) staining, and experiments were performed using an optical microscope. The histological pattern of lung tissue infected with E. coli K1 showed severe edema, pulmonary congestion, and alveolar hemorrhage with PMNs infiltrated into the lungs. As R-Pro9-3D pretreatment significantly prevented neutrophil infiltration, it efficiently restored pathological characteristics induced by E. coli K1 (FIG. 8J).

Through the above-mentioned results, it was demonstrated that R-Pro9-3D of the present invention is a peptide anti-septic that is highly effective in treating Gram-negative sepsis infections.

[Sequence Listing Free Text]

R-Pro9-3 peptide
SEQ ID NO: 1
Arg Arg Trp Ile Ala Leu Trp Leu Arg
1 5
R-Pro9-3D peptide
SEQ ID NO: 2
Arg Arg Trp Ile Ala Leu Trp Leu Arg
1 5
Pro9-3 peptide
SEQ ID NO: 3
Arg Leu Trp Leu Ala Ile Trp Arg Arg
1 5
Pro9-3D peptide
SEQ ID NO: 4
Arg Leu Trp Leu Ala Ile Trp Arg Arg
1 5

1. A nonameric peptide represented by an amino acid sequence of SEQ ID NO: 1 or its enantiomeric peptide thereof.

2. The peptide of claim 1, wherein the nonameric peptide or its enantiomeric peptide thereof is an antimicrobial peptide.

3. The peptide of claim 1, wherein all amino acids of the amino acid sequence of SEQ ID NO: 1 in the enantiomeric peptide are substituted with D-type amino acids.

4. The peptide of claim 2, wherein the antimicrobial peptide has antimicrobial activity against Gram-negative bacteria.

5. The peptide of claim 4, wherein the Gram-negative bacteria are multidrug-resistant (MDR) Gram-negative bacteria.

6. The peptide of claim 4, wherein the Gram-negative bacteria comprise one or more selected from the group consisting of Escherichia coli, Acinetobacter baumannii, Pseudomonas aeruginosa, and Klebsiella pneumonia.

7. The peptide of claim 5, wherein the multidrug-resistant (MDR) Gram-negative bacteria are carbapenem-resistant.

8. The peptide of claim 1, wherein the nonameric peptide or its enantiomeric peptide thereof has low hemolytic activity and low toxicity on mammalian cells.

9. The peptide of claim 1, wherein the nonameric peptide or its enantiomeric peptide thereof increases bacterial cell membrane permeability to damage bacterial cell membranes.

10. The peptide of claim 1, wherein the nonameric peptide or its enantiomeric peptide thereof inhibits the activity of lipopolysaccharide (LPS) via binding to LPS, which is a component of the outer membrane of Gram negative bacteria and acts as an endotoxin.

11. The peptide of claim 1, wherein the nonameric peptide or its enantiomeric peptide thereof suppresses the formation of a bacterial biofilm.

12. The peptide of claim 1, wherein the enantiomeric peptide has proteolytic stability with resistance to proteases.

13. The peptide of claim 1, wherein the nonameric peptide or its enantiomeric peptide thereof suppresses inflammation induced by LPS.

14-22. (canceled)

23. A method for preventing or treating Gram-negative bacterial infectious diseases, the method comprising: administering a pharmaceutical composition comprising the peptide of claim 1 as an active ingredient to a patient with a Gram-negative bacterial infectious disease.

24-28. (canceled)
